(19) United States
(12) Patent Application Publication
(10) Pub. No.: US 2021/0393242 A1
(43) Pub. Date: Dec. 23, 2021

(54) DEVICE AND METHODS FOR MOTION ARTIFACT SUPPRESSION IN AUSCULTATION AND ULTRASOUND DATA

(71) Applicant: EchoNous, Inc., Redmond, WA (US)

(72) Inventors: Ramachandra Pailoor, Woodinville, WA (US); Eung-Hun Kim, Bothell, WA (US); Qianying Miao, Redmond, WA (US); Nidhi Jaiswal, Redmond, WA (US); Nikolaos Pagoulatos, Kirkland, WA (US)

(21) Appl. No.: 17/350,914

(22) Filed: Jun. 17, 2021

Related U.S. Application Data
(60) Provisional application No. 63/041,569, filed on Jun. 19, 2020.

(51) Int. Cl. A61B 8/08

(52) U.S. Cl. CPC A61B 8/5276; A61B 8/5223

(57) ABSTRACT

Devices, systems, and methods are provided in which noise or artifacts generated by motion of a probe are attenuated in acquired physiological data, such as auscultation or ultrasound data. One such method includes acquiring physiological data of a patient by a handheld device. Motion of the handheld device is sensed, and a determination is made as to whether the sensed motion of the handheld device exceeds a motion threshold. The method further includes generating conditioned physiological data of the patient by attenuating a portion of the acquired physiological data in response to determining that the sensed motion of the handheld device exceeds the motion threshold.

### BACKGROUND

### Technical Field

The present application pertains to physiological sensing devices, systems and methods, and more particularly to such devices, systems and methods for acquiring auscultation and ultrasound data.

### Description of the Related Art

Ultrasound imaging is typically performed in a clinical setting, by trained ultrasound experts, utilizing ultrasound systems that are specifically designed to acquire ultrasound data. Auscultation data is typically acquired by a physician or other clinician utilizing a stethoscope. Acquisition of these different types of clinical data, i.e., ultrasound data and auscultation data, is thus conventionally performed utilizing separate pieces of equipment, and often in separate patient visits or separate environments.

Examinations of a patient using either ultrasound devices or auscultation devices generally involve positioning the device against the patient's skin while data (e.g., ultrasound or auscultation data) is acquired. During the examination, the device is typically moved around on the patient. Friction between the sensing surface of the ultrasound or auscultation device and the patient's skin can result in relatively loud noises or motion artifacts in the acquired signals.

### BRIEF SUMMARY

The present disclosure, in part, addresses a desire for physiological sensing devices that are capable of suppressing motion-related noises and artifacts from auscultation and ultrasound data. Motion of an auscultation device during acquisition of auscultation data, for example during examination of a patient, can induce noise or audible artifacts that may be objectionable to hear and in some cases may lead to misinterpretation of the auscultation data. Similarly, motion of an ultrasound device during acquisition of ultrasound data may induce undesirable noise or artifacts in an audible output, such as an audio signal associated with blood flow during Doppler ultrasound imaging.

The present disclosure provides systems, devices, and methods in which detected motion of an auscultation or ultrasound device is utilized to condition the auscultation or ultrasound data. The auscultation or ultrasound data may be conditioned, for example, by attenuating or removing motion-based artifacts from the auscultation or ultrasound data. The conditioned auscultation or ultrasound data may then be output, for example, through a speaker, headphones, or the like, and the objectionable sounds related to the motion-based artifacts may be avoided or reduced.

In some embodiments, the present disclosure provides an ultrasound device including an auscultation sensor and a motion sensor. The motion sensor is utilized to detect motion of the device. In some embodiments, when the detected motion exceeds a motion threshold, auscultation or ultrasound data acquired by the device is conditioned, e.g., by suppressing audio signals associated with the auscultation or ultrasound data for a duration of time in which the detected motion exceeds the motion threshold.

In at least one embodiment, a method is provided that includes acquiring, by a handheld device, physiological data of a patient; sensing a motion of the handheld device; determining whether the sensed motion of the handheld device exceeds a motion threshold; and generating conditioned physiological data of the patient by attenuating a portion of the acquired physiological data in response to determining that the sensed motion of the handheld device exceeds the motion threshold.

In at least one embodiment, a system is provided that includes a handheld probe, motion detection circuitry, and motion artifact suppression circuitry. The handheld probe includes a physiological sensor that acquires physiological data of a patient, and a motion sensor that senses motion of the handheld probe. The motion detection circuitry determines whether the sensed motion of the handheld probe exceeds a motion threshold. The motion artifact suppression circuitry generates conditioned physiological data of the patient by attenuating a portion of the acquired physiological data in response to the motion detection circuitry determining that the motion of the handheld probe exceeds the motion threshold.

In at least one embodiment, a system is provided that includes a probe including an auscultation sensor and an ultrasound sensor. Motion detection circuitry determines whether a motion of the probe exceeds a motion threshold. Motion artifact suppression circuitry generates conditioned physiological data by attenuating a portion of physiological data acquired by the auscultation sensor or the ultrasound sensor in response to the motion detection circuitry determining that the motion of the probe exceeds the motion threshold.

### BRIEF DESCRIPTION OF THE SEVERAL VIEWS OF THE DRAWINGS

FIG. 1 is a schematic illustration of a physiological sensing device including a probe and a handheld computing device, in accordance with one or more embodiments of the present disclosure.

FIG. 2 is a perspective view illustrating further details of the probe of the physiological sensing device shown in FIG. 1, in accordance with one or more embodiments.

FIG. 3 is an enlarged perspective view of a sensor portion of the probe shown in FIG. 2, in accordance with embodiments of the present disclosure, in accordance with one or more embodiments.

FIG. 4 is a block diagram illustrating electrical features of the physiological sensing device, in accordance with one or more embodiments.

FIG. 5 is a block diagram illustrating electrical features of the physiological sensing device, in accordance with one or more embodiments.

FIG. 6 is a waveform diagram illustrating attenuation of motion artifacts in a digital auscultation signal, in accordance with one or more embodiments.

### DETAILED DESCRIPTION

Medical ultrasound imaging (sonography) is one of the most effective methods for examining both the heart and the lungs. Ultrasound imaging provides anatomical information of the heart as well as qualitative and quantitative information on blood flow through valves and main arteries such as the aorta and pulmonary artery. One significant advantage of ultrasound imaging is that, with its high frame rate, it can provide dynamic anatomical and blood flow information which is vital for assessing the condition of the heart which is always in motion. Combined with providing blood flow information, ultrasound imaging provides one of the best available tools for assessing the structure and function of heart chambers, valves, and arteries/veins. Similarly, ultrasound imaging can assess fluid status in the body and is the best tool in assessing pericardial effusion (fluid around the heart).

In the case of lungs, ultrasound imaging provides information on the anatomical structure of the lungs with an ability to show specific imaging patterns associated with various lung diseases and an ability to assess fluid status around the lung and within individual compartments of the lung including an assessment of pericardial effusion.

Auscultation allows for assessing the physiological condition and function of organs such as the heart and lungs by capturing audible sounds that are produced by or otherwise associated with these organs. The condition and function of these organs, or other organs as the case may be, can be evaluated based on clinical information indicating how different sounds are associated with various physiological phenomena and how the sounds change for each pathological condition.

The present disclosure provides devices and methods in which detected or determined motion of a handheld probe is utilized to attenuate motion-related artifacts or noise from acquired physiological data (e.g., auscultation data or signal data associated with ultrasound imaging, including for example blood flow data obtained during Doppler ultrasound imaging). In some embodiments, the motion of the probe is detected by a motion sensor, and the detected motion is compared to a motion threshold. If the detected motion exceeds the motion threshold, the acquired physiological data may be conditioned, for example, by attenuating the data during periods of time in which the detected motion exceeds the motion threshold. Depending on circumstances and implementation of the present disclosure, attenuating data as described herein may include suppressing the data during the periods of time in which the detected motion exceeds the motion threshold.

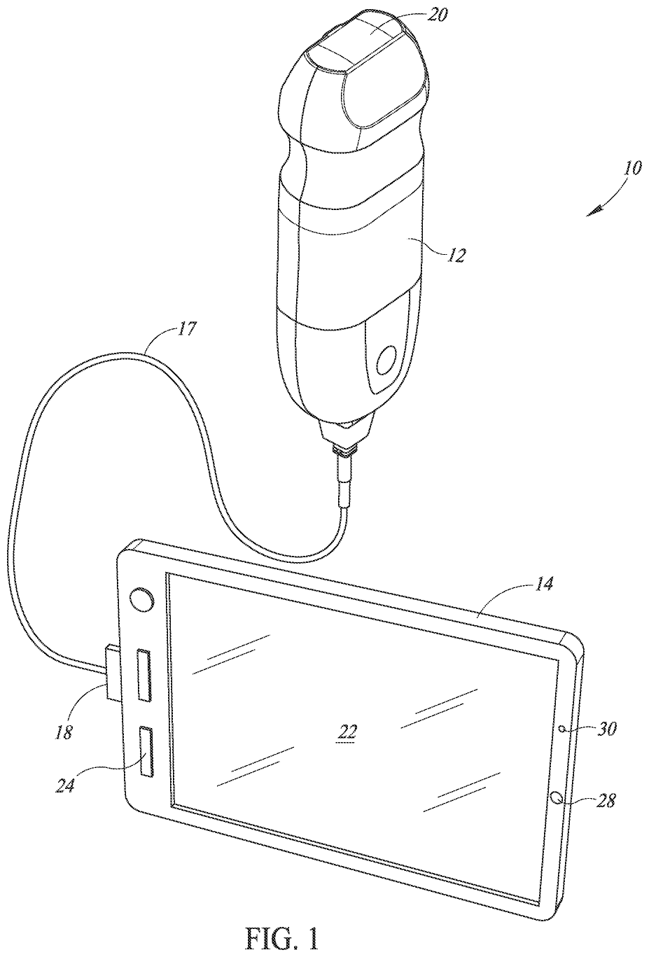

FIG. 1 is a schematic illustration of a physiological sensing device 10, in accordance with one or more embodiments of the present disclosure. The device 10 includes a probe 12 that, in the illustrated embodiment, is electrically coupled to a handheld computing device 14 by a cable 17. The cable 17 includes a connector 18 that detachably connects the probe 12 to the computing device 14. The handheld computing device 14 may be any portable computing device having a display, such as a tablet computer, a smartphone, or the like. In some embodiments, the probe 12 need not be electrically coupled to the handheld computing device 14, but may operate independently of the handheld computing device 14, and the probe 12 may communicate with the handheld computing device 14 via a wireless communication channel.

The probe 12 is configured to transmit an ultrasound signal toward a target structure and to receive echo signals returning from the target structure in response to transmission of the ultrasound signal. The probe 12 includes an ultrasound sensor 20 that, in various embodiments, may include an array of transducer elements (e.g., a transducer array) capable of transmitting an ultrasound signal and receiving subsequent echo signals.

The device 10 further includes processing circuitry and driving circuitry. In part, the processing circuitry controls the transmission of the ultrasound signal from the ultrasound sensor 20. The driving circuitry is operatively coupled to the ultrasound sensor 20 for driving the transmission of the ultrasound signal, e.g., in response to a control signal received from the processing circuitry. The driving circuitry and processor circuitry may be included in one or both of the probe 12 and the handheld computing device 14. The device 10 also includes a power supply that provides power to the driving circuitry for transmission of the ultrasound signal, for example, in a pulsed wave or a continuous wave mode of operation.

The ultrasound sensor 20 of the probe 12 may include one or more transmit transducer elements that transmit the ultrasound signal and one or more receive transducer elements that receive echo signals returning from a target structure in response to transmission of the ultrasound signal. In some embodiments, some or all of the transducer elements of the ultrasound sensor 20 may act as transmit transducer elements during a first period of time and as receive transducer elements during a second period of time that is different than the first period of time (i.e., the same transducer elements may be usable to transmit the ultrasound signal and to receive echo signals at different times).

The computing device 14 shown in FIG. 1 includes a display screen 22 and a user interface 24. The display screen 22 may be a display incorporating any type of display technology including, but not limited to, LCD or LED display technology. The display screen 22 is used to display one or more images generated from echo data obtained from the echo signals received in response to transmission of an ultrasound signal, and in some embodiments, the display screen 22 may be used to display color flow image information, for example, as may be provided in a Color Doppler imaging (CDI) mode. Moreover, in some embodiments, the display screen 22 may be used to display audio waveforms, such as waveforms representative of an acquired or conditioned auscultation signal.

In some embodiments, the display screen 22 may be a touch screen capable of receiving input from a user that touches the screen. In such embodiments, the user interface 24 may include a portion or the entire display screen 22, which is capable of receiving user input via touch. In some embodiments, the user interface 24 may include one or more buttons, knobs, switches, and the like, capable of receiving input from a user of the ultrasound device 10. In some embodiments, the user interface 24 may include a microphone 30 capable of receiving audible input, such as voice commands.

The computing device 14 may further include one or more audio speakers 28 that may be used to output acquired or conditioned auscultation signals, or audible representations of echo signals, blood flow during Doppler ultrasound imaging, or other features derived from operation of the device 10.

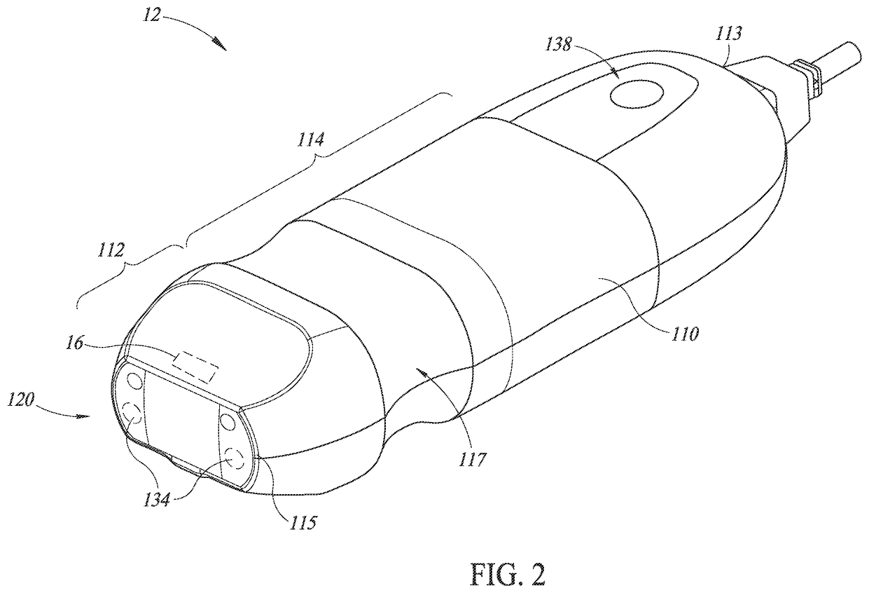

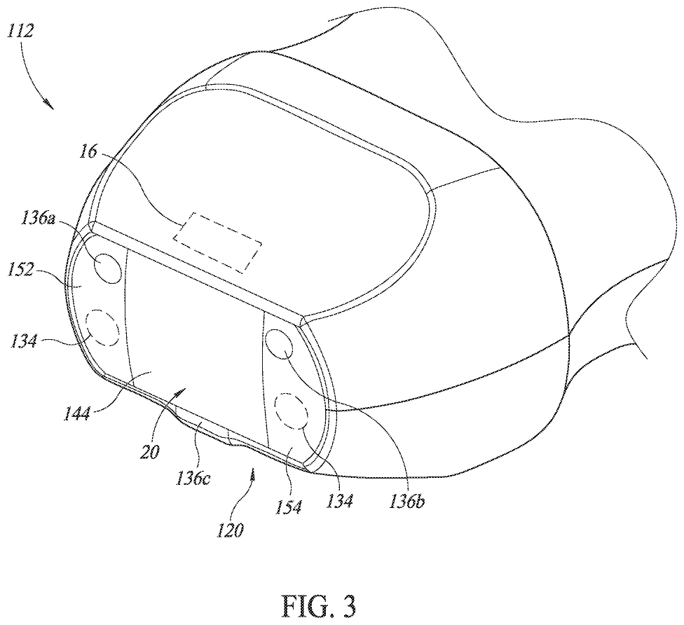

FIG. 2 is a perspective view illustrating further details of the probe 12, and FIG. 3 is an enlarged perspective view of a sensor portion of the probe 12 shown in FIG. 2, in accordance with embodiments of the present disclosure.

As shown in FIG. 2, the probe 12 includes a housing 110, which forms an external portion of the probe 12. The housing 110 includes a sensor portion 112 located near a distal end 115 of the housing 110, and a handle portion 114 located between a proximal end 113 and the distal end 115 of the housing 110. The handle portion 114 is proximally located with respect to the sensor portion 112.

The handle portion 114 is a portion of the housing 110 that is gripped by a user to hold, control, and manipulate the probe 12 during use. The handle portion 114 may include gripping features, such as one or more detents 117, and in some embodiments, the handle portion 114 may have a same general shape as portions of the housing 110 that are distal to, or proximal to, the handle portion 114.

The housing 110 surrounds internal electronic components and/or circuitry of the probe 12, including, for example, electronics such as driving circuitry, processing circuitry, oscillators, beamforming circuitry, filtering circuitry, and the like. The housing 110 may be formed to surround or at least partially surround externally located portions of the probe 12, such as a sensing surface 120 (see FIG. 3). The housing 110 may be a sealed housing, such that moisture, liquid or other fluids are prevented from entering the housing 110. The housing 110 may be formed of any suitable materials, and in some embodiments, the housing 110 is formed of a plastic material. The housing 110 may be formed of a single piece (e.g., a single material that is molded surrounding the internal components) or may be formed of two or more pieces (e.g., upper and lower halves) which are bonded or otherwise attached to one another.

As shown in FIGS. 2 and 3, the probe 12 includes a motion sensor 16. The motion sensor 16 is operable to sense a motion of the probe 12. The motion sensor 16 is included in or on the probe 12 and may include, for example, one or more accelerometers, magnetometers, or gyroscopes for sensing motion of the probe 12. For example, the motion sensor 16 may be or include any of a piezoelectric, piezoresistive, or capacitive accelerometer capable of sensing motion of the probe 12. In some embodiments, the motion sensor 16 may be a tri-axial motion sensor capable of sensing motion about any of three axes. In some embodiments, more than one motion sensor 16 is included in or on the probe 12. In some embodiments, the motion sensor 16 includes at least one accelerometer and at least one gyroscope.

The motion sensor 16 may be housed at least partially within the housing 110 of the probe 12. In some embodiments, the motion sensor 16 is positioned at or near the sensing surface 120 of the probe 12. In some embodiments, the sensing surface 120 is a surface which is operably brought into contact with a patient during an examination, such as for ultrasound imaging or auscultation sensing. The ultrasound sensor 20 and one or more auscultation sensors 134 are positioned on, at, or near the sensing surface 120, as shown.

By positioning the motion sensor 16 near the sensing surface 120 of the probe 12, and hence near the ultrasound sensor 20 and the auscultation sensors 134, the motion sensor 16 may sense the motions of the sensing surface 120 as it is used, for example, during ultrasound imaging and auscultation sensing.

As shown in further detail in FIG. 3, in some embodiments, the sensor portion 112 includes an electrocardiogram (EKG) sensor 136, which may include a plurality of EKG leads or electrodes 136a, 136b, 136c. Each of the ultrasound sensor 20, the auscultation sensors 134, and the EKG sensor 136 are positioned at the sensing surface 120 of the probe 12. In use, the sensing surface 120 may be placed in contact with a patient's skin, and the probe 12 may obtain ultrasound, auscultation, and EKG signals via the ultrasound sensor 20, the auscultation sensors 134, and the EKG sensor 136, respectively.

In some embodiments, the probe 12 includes two auscultation sensors 134 at or adjacent to the sensing surface 120, as shown. The auscultation sensors 134 may be any sensor operable to detect internal body sounds of a patient, including, for example, body sounds associated with the circulatory, respiratory, and gastrointestinal systems. For example, in some embodiments, the auscultation sensors 134 may be microphones. In some embodiments, the auscultation sensors 134 may be electronic or digital stethoscopes, and may include or otherwise be electrically coupled to amplification and signal processing circuitry for amplifying and processing sensed auscultation signals, as may be known in the relevant field.

Referring again to FIG. 2, in some embodiments, the probe 12 further includes an ambient noise sensor 138 which may be positioned at least partially within the housing 110 between the handle portion 114 and the proximal end 113 of the housing 110. The ambient noise sensor 138 may be any microphone suitable to detect ambient sounds, such as non-clinically significant sounds that may be present within an external environment in which the probe 12 is utilized during an examination. In some embodiments, the ambient noise sensor 138 senses ambient sounds, and a noise-canceled signal may be generated in which the ambient sounds are canceled from the target sounds (e.g., heart sounds of a patient) that are sensed by the auscultation sensors 134.

The ambient sounds that are sensed by the ambient noise sensor 138 may be, for example, sounds generated by electronic equipment, sounds generated by the ultrasound sensor 20 (e.g., by an ultrasound array), sounds generated by a user of the device 10, such as by movement of the user's hands, or any other sounds that may be present in an environment in which the device 10 is used.

As noted previously herein, the ultrasound sensor 20 includes a transducer array configured to transmit an ultrasound signal toward a target structure in a region of interest in the patient. The transducer array is further configured to receive echo signals returning from the target structure in response to transmission of the ultrasound signal. In various embodiments, the transducer elements of the transducer array may be arranged as elements of a phased array. Suitable phased array transducers are known in the art.

In some embodiments, the transducer array of the ultrasound sensor 20 may be a one-dimensional (1D) array or a two-dimensional (2D) array of transducer elements. The transducer array may include piezoelectric ceramics, such as lead zirconate titanate (PZT), or may be based on microelectromechanical systems (MEMS). For example, in various embodiments, the ultrasound sensor 20 may include piezoelectric micromachined ultrasonic transducers (PMUT), which are microelectromechanical systems (MEMS)-based piezoelectric ultrasonic transducers, or the ultrasound sensor 20 may include capacitive micromachined ultrasound transducers (CMUT) in which the energy transduction is provided due to a change in capacitance.

The ultrasound sensor 20 may further include an ultrasound focusing lens 144, which may be positioned over the transducer array, and which may form a part of the sensing surface 120. The focusing lens 144 may be any lens operable to focus a transmitted ultrasound beam from the transducer array toward a patient and/or to focus a reflected ultrasound beam from the patient to the transducer array. The ultrasound focusing lens 144 may have a curved surface shape in some embodiments. The ultrasound focusing lens 144 may have different shapes, depending on a desired application, e.g., a desired operating frequency, or the like. The ultrasound focusing lens 144 may be formed of any suitable material, and in some embodiments, the ultrasound focusing lens 144 is formed of a room-temperature-vulcanizing (RTV) rubber material.

The EKG sensor 136 may be any sensor that detects electrical activity, e.g., of a patient's heart, as may be known in the relevant field. For example, the EKG sensor 136 may include any number of electrodes 136a, 136b, 136c, which in operation are placed in contact with a patient's skin and are used to detect electrical changes in the patient that are due to the heart muscle's pattern of depolarizing and repolarizing during each heartbeat.

As shown in FIG. 3, the EKG sensor 136 may include a first electrode 136a that is positioned adjacent to a first side of the ultrasound sensor 20 (e.g., adjacent to the left side of the ultrasound focusing lens 144, as shown), and a second electrode 136b that is positioned adjacent to a second side of the ultrasound sensor 20 that is opposite to the first side (e.g., adjacent to the right side of the ultrasound focusing lens 144, as shown). The EKG sensor 136 may further include a third electrode 136c that is positioned adjacent to a third side of the ultrasound sensor 20 (e.g., adjacent to the lower side of the ultrasound focusing lens 144, as shown). In some embodiments, each of the first, second, and third electrodes 136a, 136b, 136c have different polarities. For example, the first electrode 136a may be a positive (+) electrode, the second electrode 136b may be a negative (−) electrode, and the third electrode 136c may be a ground electrode.

In some embodiments, first and second membranes 152, 154 are positioned adjacent to opposite sides of the ultrasound sensor 20 and may form a part of the sensing surface 120. The membranes 152, 154 may be formed of any suitable material, and in some embodiments, the membranes 152, 154 are formed of a room-temperature-vulcanizing (RTV) rubber material. In some embodiments, the membranes 152, 154 are formed of a same material as the ultrasound focusing lens 144.

The membranes 152, 154 may be positioned in front of (i.e., distally with respect to) the auscultation sensors 134. In some embodiments, the auscultation sensors 134 are spaced apart from the membranes 152, 154 by gaps, which may be air gaps. The ambient noise sensor 138 may similarly be positioned on or in the housing 110 with a membrane positioned over the ambient noise sensor 138.

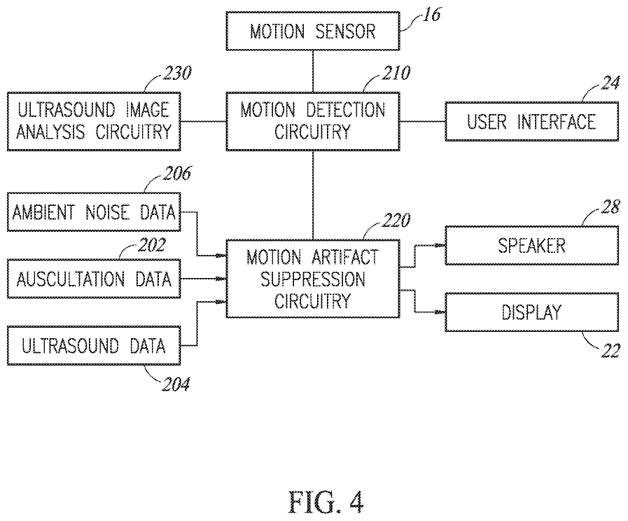

FIG. 4 is a block diagram illustrating electrical features of the device 10, which may be utilized to condition received signals to attenuate (reduce, suppress, or remove) motion artifacts in accordance with one or more embodiments.

As shown in FIG. 4, the motion sensor 16 is communicatively coupled (e.g., electrically coupled) to motion detection circuitry 210. The motion detection circuitry 210 is communicatively coupled to motion artifact suppression circuitry 220. In some embodiments, ultrasound image analysis circuitry 230 is included in the device 10. Each of the motion detection circuitry 210, the motion artifact suppression circuitry 220, and the ultrasound image analysis circuitry 230 may be or include one or more programmed processors that operate in accordance with computer-executable instructions that, in response to execution, cause the programmed processors to perform various actions, such as any of the functions as described herein with respect to the motion detection circuitry 210, the motion artifact suppression circuitry 220, and the ultrasound image analysis circuitry 230. In some embodiments, the motion detection circuitry 210, the motion artifact suppression circuitry 220, and the ultrasound image analysis circuitry 230 may be implemented, at least in part, in a programmed processor or an application specific integrated circuit (ASIC) configured to provide the motion detection, signal conditioning, and ultrasound image analysis functions described herein.

In some embodiments, the motion detection circuitry 210, the motion artifact suppression circuitry 220, and the ultrasound image analysis circuitry 230 may include or otherwise be communicatively coupled to computer-readable memory, which may store computer-executable instructions that, in part, are executable by the motion detection circuitry 210, the motion artifact suppression circuitry 220, and the ultrasound image analysis circuitry 230 and cause the motion detection circuitry 210, the motion artifact suppression circuitry 220, and the ultrasound image analysis circuitry 230 to perform the various actions described herein.

In some embodiments, one or more of the motion detection circuitry 210, the motion artifact suppression circuitry 220, and the ultrasound image analysis circuitry 230 are housed within the housing 110 of the probe 12. For example, the motion detection circuitry 210, the motion artifact suppression circuitry 220, and the ultrasound image analysis circuitry 230 may be provided as circuitry (e.g., one or more ASICs) that is mounted on and electrically coupled to a circuit board, such as a printed circuit board (PCB), located within the housing 110 of the probe 12.

In other embodiments, one or more of the motion detection circuitry 210, the motion artifact suppression circuitry 220, and the ultrasound image analysis circuitry 230 are provided within the computing device 14 and configured to receive and process signals from the motion sensor 16, the ultrasound sensor 20, and the auscultation sensors 134 of the probe 12.

The motion detection circuitry 210 is configured to detect motion of the probe 12 which may be associated with or which may cause motion-related artifacts in acquired auscultation data 202 or ultrasound data 204. In some embodiments, the motion detection circuitry 210 is configured to compare the detected motion of the probe 12 with a motion threshold. If the detected motion of the probe 12 exceeds the motion threshold, the motion detection circuitry 210 outputs a signal to the motion artifact suppression circuitry 220, and the motion artifact suppression circuitry 220 attenuates (suppresses, reduces, or removes) motion artifacts in the received auscultation data 202 or ultrasound data 204 in response to receiving the signal from the motion detection circuitry 210. The motion threshold may be stored, for example, in memory within or communicatively coupled to the motion detection circuitry 210.

In some embodiments, the motion detection circuitry 210 may compare the detected motion of the probe 12 with a plurality of motion thresholds and may determine whether the motion of the probe 12 exceeds any of the plurality of motion thresholds. For example, in some embodiments, the motion threshold may include one or more of an acceleration threshold and a velocity threshold. Sudden or jerking movements of the probe 12 which exceed the acceleration threshold may cause motion-related artifacts in the auscultation data 202 or the ultrasound data 204 (e.g., motion-related artifacts may be present in an audio signal associated with blood flow during Doppler ultrasound imaging). Motions of the probe 12 that are smooth (e.g., without excessive accelerations) but at a high velocity can similarly result in unwanted motion artifacts, and such motions may be detected by comparison of the sensed motion of the probe 12 with a velocity threshold.

In various embodiments, the motion detection circuitry 210 detects motion of the probe 12 based on motion sensed by the motion sensor 16 or based on analysis of acquired ultrasound images by the ultrasound image analysis circuitry 230.

In some embodiments, the motion detection circuitry 210 detects motion of the probe 12 based on a motion signal received from the motion sensor 16. As described previously herein, the motion sensor 16 may include one or more of an accelerometer, a magnetometer, and a gyroscope configured to sense motions of the probe 12 during use. The motion sensor 16 outputs the motion signal indicative of the sensed motion of the probe 12, and the motion detection circuitry 210 compares the motion signal with one or more motion thresholds to determine whether the sensed motion exceeds the one or more motion thresholds.

In some embodiments, the motion detection circuitry 210 detects motion of the probe 12 based on an analysis of ultrasound images acquired by the device 10 during use, such as during examination of a patient. For example, in some embodiments, the ultrasound image analysis circuitry 230 senses or detects motion of the probe 12 by analyzing the acquired ultrasound images. The ultrasound image analysis circuitry 230 may perform a frame-to-frame analysis of the acquired ultrasound images to detect changes in pose or position of the probe 12 and thereby detect motions of the probe 12. Any suitable image analysis technique may be implemented by the ultrasound image analysis circuitry 230 to detect motion of the probe 12. In some embodiments, the ultrasound image analysis circuitry 230 implements image processing or recognition techniques to recognize an imaged structure (e.g., a boundary of an organ, such as the heart) within the acquired ultrasound images, and to determine a distance at which the imaged structure moves between successive ultrasound image frames. The ultrasound image analysis circuitry may correlate the distance at which the imaged structure moves between frames with motions of the probe 12, and in some embodiments, the ultrasound image analysis circuitry 230 outputs a signal to the motion detection circuitry 210 indicative of the motions of the probe 12. The motion detection circuitry 210 may thus compare the motions of the probe 12 (e.g., as indicated by the output of the ultrasound image analysis circuitry 230) to the motion threshold to determine whether the motions of the probe 12 exceed the motion threshold. In some cases, embodiments of the device 10 that detect motion of the probe 12 based on analysis of acquired ultrasound images may operate without a motion sensor, and thus exclude the motion sensor 16 described above.

If the detected motion of the probe 12 exceeds the motion threshold, the motion detection circuitry 210 outputs an excessive motion signal to the motion artifact suppression circuitry 220. In response to receiving the excessive motion signal, the motion artifact suppression circuitry 220 attenuates motion artifacts in the received auscultation data 202 or ultrasound data 204. In some embodiments, the motion detection circuitry 210 outputs the excessive motion signal for only a duration at which the detected motion of the probe 12 exceeds the motion threshold, and the excessive motion signal is not output for all other times (e.g., when the detected motion of the probe 12 is below the motion threshold).

The motion artifact suppression circuitry 220 may include any signal processing circuitry suitable to attenuate the received auscultation data 202 or ultrasound data 204 in response to receiving the excessive motion signal, including, for example, adjustable gain amplifiers, filtering circuitry, or the like. In some embodiments, the motion artifact suppression circuitry 220 attenuates motion-artifacts in the received auscultation data 202 or ultrasound data 204 by implementing a band-pass filter in response to receiving the excessive motion signal, so that only frequencies within a particular band are passed to the audio speakers 28 or the display 22. In some embodiments, in response to receiving the excessive motion signal, the motion artifact suppression circuitry 220 automatically decreases a volume of the speakers 28 or automatically decreases a gain of a gain controller for displaying waveforms of the auscultation data 202 or ultrasound data 204 on the display 22.

In some embodiments, the motion artifact suppression circuitry 220 includes one or more switching devices, such as transistors, which are operable to selectively pass the auscultation data 202 or the ultrasound data 204 to the audio speakers 28 or the display 22. For example, the switching devices of the motion artifact suppression circuitry 220 may be controlled based on the excessive motion signal, and the motion artifact suppression circuitry 220 may block or otherwise impede the auscultation data 202 or ultrasound data 204 from being output to the speakers 28 or display 22 when the excessive motion signal is received from the motion detection circuitry 210.

In some embodiments, the motion sensor 16 includes two or more motion sensors, each of which may have an associated motion threshold. For example, in an embodiment, the probe 12 includes an accelerometer and a gyroscope. The motion information received from the accelerometer may be compared to an accelerometer motion threshold, and the motion information received from the gyroscope may be compared to a gyroscope motion threshold.

The motion artifact suppression circuitry 220 may attenuate motion artifacts in the auscultation data 202 or the ultrasound data 204 based on the motion information received from either or both of the accelerometer and the gyroscope. For example, in some embodiments, the motion artifact suppression circuitry 220 may attenuate motion artifacts in the auscultation data 202 or the ultrasound data 204 in response to the motion information received from the accelerometer being greater than the accelerometer motion threshold, even if the motion information received from the gyroscope is below the gyroscope motion threshold. Similarly, in some embodiments, motion artifact suppression circuitry 220 may attenuate motion artifacts in the auscultation data 202 or the ultrasound data 204 in response to the motion information received from the gyroscope being greater than the gyroscope motion threshold, even if the motion information received from the accelerometer is below the accelerometer motion threshold.

In some embodiments, the motion artifact suppression circuitry 220 may attenuate motion artifacts in the auscultation data 202 or the ultrasound data 204 only in response to motion information received from both the accelerometer and the gyroscope being greater than the accelerometer motion threshold and the gyroscope motion threshold, respectively.

In some embodiments, the motion detection circuitry 210 is coupled to the user interface 24. The user interface 24 may receive user input, for example, as touch inputs on the display 22, or as user input via one or more buttons, knobs, switches, and the like. In some embodiments, the user interface 24 may receive audible user input, such as voice commands received by a microphone 30 of the computing device 14.

In some embodiments, one or more of the motion thresholds may be adjustable, for example, based on user input. For example, in some embodiments, a user may adjust levels of the motion thresholds by providing input via the user interface 24. Adjustable motion thresholds allow the user to tune out or otherwise attenuate motion artifacts based on their personal preference. Moreover, the adjustable motion thresholds allow the user to set the motion thresholds differently depending on a type of examination being performed. For example, the noises associated with motion artifacts may be more or less significant depending on what type of exam is being performed, e.g., depending on what sounds the user is listening for.

Further, as shown in FIG. 4, ambient noise data 206 may be provided to the motion artifact suppression circuitry 220. The ambient noise data 206 may be provided, for example, from the ambient noise sensor 138. In some embodiments, the motion artifact suppression circuitry 220 includes noise-canceling circuitry configured to receive the auscultation data 202 sensed by the auscultation sensors 134 and the ambient noise data 206 sensed by the ambient noise sensor 138. The motion artifact suppression circuitry 220 generates a noise-canceled auscultation signal in which the ambient noise data 206 is canceled from the auscultation data 202, thus removing ambient noise from the auscultation signal that is output to the speakers 28 or the display 22. The motion artifact suppression circuitry 220 may generate the noise-canceled auscultation signal by any suitable techniques. In some embodiments, the motion artifact suppression circuitry 220 may generate a cancelation waveform that is a negative of the ambient noise signal sensed by the ambient noise sensor 138, and may mix the cancelation waveform with the auscultation signal waveform sensed by the auscultation sensors 134 in order to produce the noise-canceled auscultation signal data.

The motion artifact suppression circuitry 220 may further include or otherwise be coupled to audio processing circuitry for processing the auscultation data 202, the ultrasound data 204, and the ambient noise data 206, including, for example, filters, amplifiers, preconditioning and digitization circuitry, and the like.

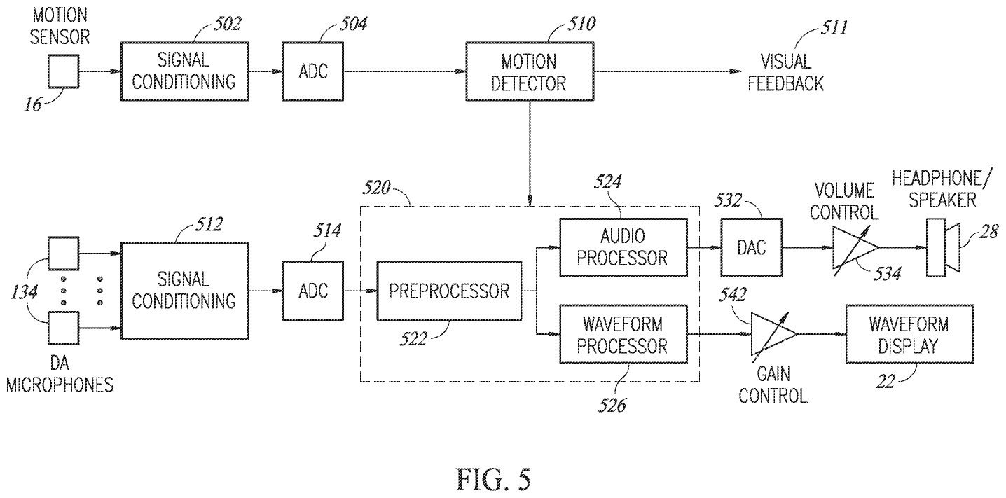

FIG. 5 is a block diagram illustrating electrical features of the device 10, which may be utilized to condition received signals to attenuate (that is, to reduce, suppress, or remove) motion artifacts in accordance with one or more embodiments. FIG. 5 is similar to the block diagram shown in FIG. 4, with additional details illustrated.

As shown in FIG. 5, the motion sensor 16 outputs the motion signal indicative of sensed motion of the probe 12 to signal conditioning circuitry 502 and analog-to-digital converter (ADC) 504. The signal conditioning circuitry 502 may include any circuitry for limiting the bandwidth of the motion signal to an acceptable or suitable range as may be desired depending on design considerations, such as any filter circuitry, amplifiers, or the like. The ADC 504 converts the analog signal to a digital signal, which is then provided to the motion detector 510.

The motion detector 510 may be any or include any motion detection circuitry, including for example the motion detection circuitry 210 previously described herein with respect to FIG. 4. For example, the motion detector 510 may include circuitry configured to compare the received motion signal with a motion threshold. In some embodiments, the motion detector 510 includes or otherwise accesses software instructions for determining whether the received motion signal indicates motion that exceeds the motion threshold. In some embodiments, the motion detector 510 implements a configurable motion threshold, which may be adjustable as previously described herein. The motion detector 510 outputs an excessive motion signal to the motion artifact suppression circuitry 520 when the sensed motion exceeds the motion threshold, and the motion artifact suppression circuitry then conditions or otherwise suppresses motion artifacts from the auscultation signals sensed by the auscultation sensors 134.

In some embodiments, a feedback signal 511 (which may be referred to herein as feedback 511) is provided to the user indicating, for example, that the detected motion exceeds the motion threshold. The feedback 511 may be visual feedback that is displayed, for example, on the display 22; however, embodiments are not limited thereto. In various embodiments, the feedback 511 may include auditory feedback, haptic feedback, or any other feedback perceptible to a user of the device 10 which indicates that motion of the probe 12 meets or exceeds a threshold motion.

The auscultation sensors 134 output sensed auscultation signals to signal conditioning circuitry 512 and ADC 514. The signal conditioning circuitry 512 may include any circuitry for limiting the bandwidth of the auscultation signals to an acceptable or suitable range as may be desired depending on design considerations, such as any filter circuitry, amplifiers, or the like. The ADC 514 converts the auscultation signals to digital signals, which are then provided to the motion artifact suppression circuitry 520. In some embodiments, the signal conditioning circuitry 512 combines the auscultation signals received from each of the auscultation sensors 134 to increase the signal-to-noise ratio (SNR), and outputs the combined signal to the ADC 514. The auscultation signals may be combined directly or, in some embodiments, the auscultation signals may be combined with a selected delay between the auscultation signals to provide an enhanced SNR of the combined signal. Combining the auscultation signals from the auscultation sensors 134 has the effect of reducing noise in the auscultation signals, as noise is typically random. That is, noise is typically uncorrelated so that noise in the auscultation signal from one of the auscultation sensors 134 is often not present in the auscultation signal from the other auscultation sensor 134. As such, combining the auscultation sensors 134 has the effect of increasing the SNR.

In some embodiments, the signal conditioning circuitry 512 selects only the auscultation signal output from one of the auscultation sensors 134, and the selected auscultation signal is provided to the ADC for further processing while the non-selected auscultation signals are canceled or otherwise blocked from further processing. The selected auscultation signal may be an auscultation signal provided from the auscultation sensors 134 which has the lowest or least significant noise component, while the blocked signal has a larger or largest noise component.

The ADC 514 converts the analog auscultation signal to a digital signal, which is then provided to the motion artifact suppression circuitry 520. The motion artifact suppression circuitry 520 may include any of the features or functionalities of the motion artifact suppression circuitry 220 previously described herein with respect to FIG. 4.

In some embodiments, the motion artifact suppression circuitry 520 includes a preprocessor 522. The preprocessor 522 may perform any of the functions described previously herein with respect to the motion artifact suppression circuitry 220 shown in FIG. 4. For example, the preprocessor 522 may be configured to attenuate motion-related artifacts or noise from the auscultation signals received from the auscultation sensors 134. In some embodiments, the preprocessor 522 is further configured to cancel ambient noise from the auscultation signals, for example, utilizing ambient noise sensed by the ambient noise sensor 138.

The motion artifact suppression circuitry 520 may further include audio processing circuitry 524 and waveform processing circuitry 526. The audio processing circuitry 524 may include any suitable circuitry for driving the headphones or speakers 28. Such circuitry may include any sample rate conversion circuitry for matching the sampling rate used by the digital-to-analog converter (DAC) 532. The DAC 532 converts the analog conditioned auscultation signal output from the motion artifact suppression circuitry 520 into a digital signal, which is provided to a volume controller 534 and then output to the headphones or speakers 28. The volume controller 534 may include any circuitry suitable to adjust or otherwise control the volume of the audio signal output by the headphones or speakers 28, and in some embodiments, the volume controller 534 is controllable by the user.

The motion artifact suppression circuitry 520 further includes waveform processing circuitry 526. The waveform processing circuitry 526 includes any suitable circuitry for preparing the data or signals used for rendering the waveform (e.g., the conditioned or motion artifact attenuated auscultation signal) on the display 22. In some embodiments, additional processing is performed by the waveform processing circuitry 526, for example, to visually highlight an abnormality in the waveform that is output for display on the display 22. For example, the waveform processing circuitry 526 may cause the display 22 to display the auscultation waveform with a particular color (or any other visually perceptible indication) in regions where the auscultation waveform has been attenuated due to motion artifacts related to motions of the probe 12 that exceed the motion threshold.

In some embodiments, the waveform processing circuitry 526 includes circuitry configured to automatically scale the conditioned auscultation signal so the waveform is displayed in a visually pleasing manner. For example, the scale of the displayed waveform may be dynamically adjusted based on the parameters of the waveform to be displayed (e.g., the amplitude of the waveform).

In some embodiments, the waveform processing circuitry 526 outputs the auscultation waveform including the noise or motion artifacts, while the audio processing circuitry 524 outputs only the noise-suppressed signal. For example, the waveform processing circuitry 526 may output the auscultation waveform including the portions that have motion-related artifacts, but such portions may be visually different (e.g., indicated in a different color, or other indication) from the portions that are not associated with excessive probe motion.

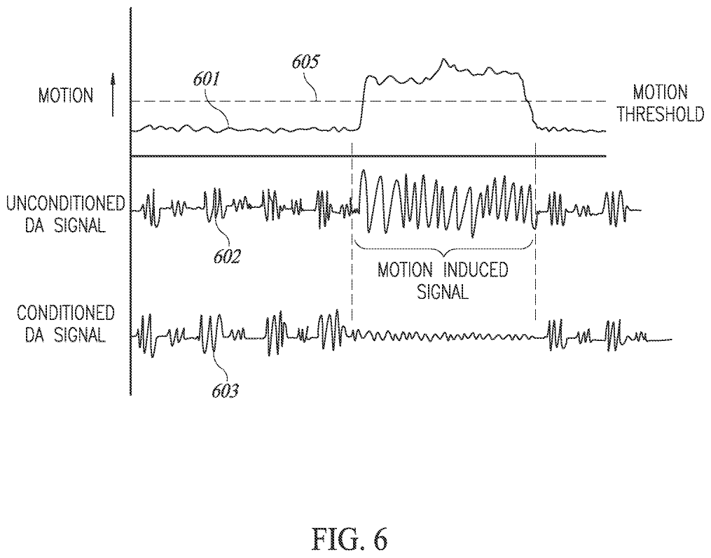

FIG. 6 is a waveform diagram illustrating attenuation of motion artifacts in a digital auscultation signal, in accordance with one or more embodiments of the present disclosure. As shown in FIG. 6, a motion signal 601, an unconditioned digital auscultation signal 602, and a conditioned digital auscultation signal 603 are temporally correlated or synchronized with one another. The motion signal 601 may be, for example, a motion signal output by the motion sensor 16. The unconditioned digital auscultation signal 602 may be, for example, the auscultation data 202 shown in FIG. 4 which may be output by the auscultation sensors 134. The conditioned digital auscultation signal 603 may be, for example, an output of the motion artifact suppression circuitry 220 which is provided to the speakers 28 or the display 22.

The waveform diagram of FIG. 6 illustrates an example of how detected motion can be used to condition the digital auscultation signal 603 which may be presented to the user, e.g., on the display 22. As shown in FIG. 6, when the detected motion indicated by the motion signal 601 exceeds the motion threshold 605 (e.g., which may be set to a level at which motion induced artifacts generally become objectionable), the conditioned digital auscultation signal 603 is attenuated or faded out (e.g., by the motion artifact suppression circuitry 220) and when it falls below the motion threshold 605 it is faded in.

Embodiments of the present disclosure provide several advantages. For example, as discussed herein, by attenuating (reducing, suppressing, or removing) noise associated with motion of a probe during examination of a patient, the user is not subjected to loud motion-related noises which can be objectionable to hear and which may obscure or lead to misinterpretation of the signals of interest. The signals of interest may be auscultation signals, and in some embodiments, the signals of interest may be audible signals associated with blood flow in Doppler ultrasound imaging.

The various embodiments described above can be combined to provide further embodiments. These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method, comprising:
acquiring, by a handheld device, physiological data of a patient;
sensing a motion of the handheld device;
determining whether the sensed motion of the handheld device exceeds a motion threshold; and
generating conditioned physiological data of the patient by attenuating a portion of the acquired physiological data in response to determining that the sensed motion of the handheld device exceeds the motion threshold.

2. The method of claim 1, further comprising:
outputting the conditioned physiological data to a speaker.

3. The method of claim 1, further comprising:
displaying the conditioned physiological data on a display of a computer device.

4. The method of claim 3 wherein the displaying the conditioned physiological data on the display of the computer device includes displaying the attenuated portion of the acquired physiological data in a first color, and displaying a non-attenuated portion of the acquired physiological data in a second color that is different than the first color.

5. The method of claim 1 wherein the sensing the motion of the handheld device includes sensing the motion of the handheld device by at least one of an accelerometer or a gyroscope.

6. The method of claim 1 wherein the acquiring physiological data of a patient includes acquiring auscultation data of the patient, and wherein the generating conditioned physiological data includes generating conditioned auscultation data.

7. The method of claim 1 wherein the acquiring physiological data of a patient includes acquiring blood flow data of the patient through ultrasound imaging, and wherein the generating conditioned physiological data includes generating conditioned blood flow data by attenuating a portion of the acquired blood flow data.

8. The method of claim 1, further comprising:
outputting a feedback signal that indicates the sensed motion of the handheld device meets or exceeds the motion threshold.

9. A system, comprising:
a handheld probe, including at least one physiological sensor configured to acquire physiological data of a patient;
motion detection circuitry configured to determine whether a motion of the handheld probe exceeds a motion threshold; and
motion artifact suppression circuitry communicatively coupled to the at least one physiological sensor and the motion detection circuitry, the motion artifact suppression circuitry configured to generate conditioned physiological data of the patient by attenuating a portion of the acquired physiological data in response to the motion detection circuitry determining that the motion of the handheld probe exceeds the motion threshold.

10. The system of claim 9 wherein handheld probe further includes a motion sensor configured to sense the motion of the handheld probe, the motion sensor including at least one of an accelerometer or a gyroscope.

11. The system of claim 9 wherein the handheld probe further includes an ambient noise sensor configured to sense ambient noise, and the motion artifact suppression circuitry is configured to generate the conditioned physiological data by canceling the ambient noise from the acquired physiological data of the patient.

12. The system of claim 9 wherein the handheld probe includes a housing having a sensor portion at a distal end of the housing, and a handle portion between a proximal end and the distal end of the housing,
wherein the at least one physiological sensor includes a first auscultation sensor positioned at least partially within the sensor portion of the housing.

13. The system of claim 12 wherein the at least one physiological sensor further includes an ultrasound sensor positioned at least partially within the sensor portion of the housing.

14. The system of claim 13 wherein the at least one physiological sensor further includes a second auscultation sensor positioned at least partially within the sensor portion of the housing, the first and second auscultation sensors spaced laterally apart from one another by the ultrasound sensor.

15. The system of claim 9, further comprising:
a computing device communicatively coupled to the handheld probe, the computing device including a display configured to display the attenuated portion of the acquired physiological data in a first color, and display a non-attenuated portion of the acquired physiological data in a second color that is different than the first color.

16. The system of claim 15 wherein the motion artifact suppression circuitry is located within the computing device.

17. A system, comprising:
a probe including an auscultation sensor and an ultrasound sensor;
motion detection circuitry configured to determine whether a motion of the probe exceeds a motion threshold; and
motion artifact suppression circuitry communicatively coupled to the motion detection circuitry and at least one of the auscultation sensor or the ultrasound sensor, the motion artifact suppression circuitry configured to generate conditioned physiological data by attenuating a portion of physiological data acquired by the at least one of the auscultation sensor or the ultrasound sensor in response to the motion detection circuitry determining that the motion of the probe exceeds the motion threshold.

18. The system of claim 17 wherein the probe further includes a motion sensor configured to sense the motion of the probe, wherein the motion detection circuitry is configured to determine whether the sensed motion of the probe exceeds the motion threshold.

19. The system of claim 17, further comprising ultrasound image analysis circuitry configured to detect the motion of the probe based on ultrasound images acquired by the ultrasound sensor.

20. The system of claim 17, further comprising a display communicatively coupled to the motion artifact suppression circuitry, the display configured to display the conditioned physiological data.
